(19) United States
(12) Patent Application Publication
(10) Pub. No.: US 2025/0134614 A1
(43) Pub. Date: May 1, 2025

(54) DISPOSABLE STERILE PROTECTIVE COVER FOR OPTICAL DEVICES USED IN OPHTHALMOLOGY

(71) Applicant: Arkadiy Farberov, Novato, CA (US)

(72) Inventor: Arkadiy Farberov, Novato, CA (US)

(21) Appl. No.: 18/386,230

(22) Filed: Nov. 1, 2023

(51) Int. Cl. A61B 50/00

(52) U.S. Cl. CPC A61B 50/00

(57) ABSTRACT

The disposable sterile protective cover is made from a thin resilient material suitable for snugly fitting over the body of an ophthalmic optical instrument such as a gonioscopic lens. The cover has a cup-shaped body tapered in the downward direction from an open upper end to an open lower end. The structure prevents physical contact between the patient's eye and the surface of the optical instrument during an ophthalmic procedure. This is achieved by providing the cover with an annular shoulder on the inner surface for stopping the optical instrument from moving to the contact with the patient's eye and for maintaining an air gap between the facing surfaces of the optical instrument and the patient's eye. Reliability of the gap can be further ensured by forming projections on the lower endface of the protective cover.

### FIELD OF THE INVENTION

The present invention relates to optical devices used in ophthalmology for intraocular observation, diagnosis, treatment, or the like, especially for angled portions, of an eyeball. In particular, the invention relates to a disposable, sterile protective cover for protecting eye-contacting lenses of aforementioned devices from physical contact with a patient's eye during intraocular observation or treatment. The use of such a disposable cover eliminates the sterilization process.

### BACKGROUND OF THE INVENTION

Diagnostic and therapeutic lenses are commonly used in ophthalmology for evaluation and treatment of the internal portions of the eye. In order to effect treatment for a variety of ocular disorders, an ophthalmologist must first see the inner structure of the eye. After an appropriate examination is completed, a laser delivery system may be used, e.g., to thermally heat the appropriate anatomical structures within the eye with laser light. Prior technology often includes a slit lamp biomicroscope used in conjunction with specialized ophthalmic instruments (devices) and special eye-contacting lenses. These lenses are designed to view different anatomical areas within the eye. The patient's pupil is usually pharmacologically dilated, topical anesthesia is placed on the eye, a coupling agent is placed on the posterior surface of the lens, and the examiner holds the lens over the surface of the eye.

During use, the instruments of the aforementioned type are brought in direct contact with the cornea of the patient's eye. In order to prevent transfer of infections from patient to patient, such optical instruments, especially the eye-contacting portions thereof, must be properly sterilized before use. The sterilization procedure most widely used in practice is treatment in an autoclave. However, since sterilization in an autoclave is a high-temperature procedure, such treatment may be undesirable for optical instruments that have optical materials, bondings, and coatings degradable under the effect of high temperatures.

Devices used in ophthalmology for intraocular observations include a line of wide-angle lenses, gonio lenses, capsulatomy and iridectomy lenses, laser photocoagulation lenses, etc.

Observation of the anterior chamber and especially its angled areas, which are difficult or impossible to see with the use of some conventional optical means, is very important for diagnosis of eye diseases. For example, the classification of glaucoma relies heavily on knowledge of anterior segment anatomy, particularly that of the anterior chamber angle.

The anterior chamber of a human eye is commonly evaluated during slit lamp biomicroscopy, but the chamber angle is hidden from ordinary view because of total internal reflection of light rays emanating from the angled structures. In other words, without gonioscopy, additional diagnostic clues of disease are forever hidden from ordinary view. Additional effort, skill, and patient cooperation are required to view the normally concealed chamber angle by either indirect (angled structures viewed through a mirror) or direct (angled structures viewed directly) gonioscopic techniques. In other words, without gonioscopy, it is impossible to classify glaucoma properly.

An example of a typical, solid, i.e., monolithic, gonioscope is shown in U.S. Pat. No. 6,767,098 issued in 2004 to Ph. Erickson, et al. The gonioscope has an optically transparent body, the distal end has a viewing surface preferably oriented perpendicularly to the optical axis of the body, the proximal end of the prism has a concave surface with a curvature similar to the curvature of a patient's cornea. The proximal end has at least one planar surface extending outwardly and distally from a location adjacent to the periphery of the concave surface. The body has an index of refraction that provides total internal reflection to a viewer looking through the viewing surface even when the planar surface is at least partially moistened with a fluid.

Common to wide-angle lenses, gonio lenses, capsulatomy and iridectomy lenses, and laser photocoagulation lenses is the provision of an eye-contacting lens that during observation or surgical treatment is maintained in physical contact with the eye cornea. Such optical devices require sterilization after use, especially with regard to the eye-contacting surfaces and especially when the optical device is intended for surgical use.

A protective cover for an optical device such as a gonioscope, capsulatomy lens, wide-angle lens, or any other lens having an eye-contacting surface is known in the art and described in U.S. Pat. No. 8,011,504B1 issued on Sep. 6, 2011 to A. Farberov, is a cover made sterile and disposable for allowing multiple use of the protected optical device. The cover is made from a soft material biologically acceptable for contact with a human eye. According to one aspect, the gonioscope protective cover is used merely for physically protecting the eye-contacting surface from direct contact with the eye. According to another aspect, the gonioscope protective cover combines the protective function with an optical function that changes the direction of light so that it becomes possible to observe the light emitted from the pupil at angles that are close to the angle of full internal reflection from the pupil medium.

### SUMMARY OF THE INVENTION

The present invention is aimed at further improvement of the disposable sterile protective cover for an optical device and at simplification of the device over one disclosed in U.S. Pat. No. 8,011,504B1.

The disposable sterile protective cover of the present invention is made from a thin resilient material suitable for snugly fitting over the body of an ophthalmic optical instrument such as a gonioscopic lens. The cover has a cup-shaped body tapered in the downward direction from an open upper end to an open lower end. The main idea of the invention is to prevent a physical contact between the patient's eye and the surface of the optical instrument during an ophthalmic procedure. This is achieved by providing an air gap between the facing surfaces of the optical instrument and the patient's eye. The air gap should have a thickness sufficient for preventing any possibility of transmitting an infection from the lower endface of the tool to the eye and, vice versa, from the eye to the tool, so that there is no need in a frequent sterilization of the tool. A reliable gap is provided by stopping the tool at a certain distance from the lower endface of the cover, i.e., above the eye surface in the operation position of the tool. A reliability of the gap can be ensured by forming an annular shoulder on the inner surface of the protective can at a certain distance from the lower endface of the protective cover. To provide a reliable air gap, projections can be made on the lower endface of the cover.

### BRIEF DESCRIPTION OF THE DRAWINGS

FIG. 1 is s vertical sectional view of the disposable sterile protective cover of the invention.

FIG. 2 is a view of the device of FIG. 1 in the direction of arrow A in FIG. 1.

FIG. 3 is a vertical sectional view of the disposable sterile protective cover of the invention in accordance with another modification.

FIG. 4 is a view of the device of FIG. 3 in the direction of arrow B in FIG. 3.

### DETAILED DESCRIPTION OF THE INVENTION

The present invention relates to optical devices used in ophthalmology for intraocular observation, diagnosis, treatment, or the like, especially for angled portions, of an eyeball. In particular, the invention relates to a disposable, sterile protective cover for protecting eye-contacting lenses of aforementioned devices from physical contact with a patient's eye during intraocular observation or treatment. The use of such a disposable cover eliminates the sterilization process.

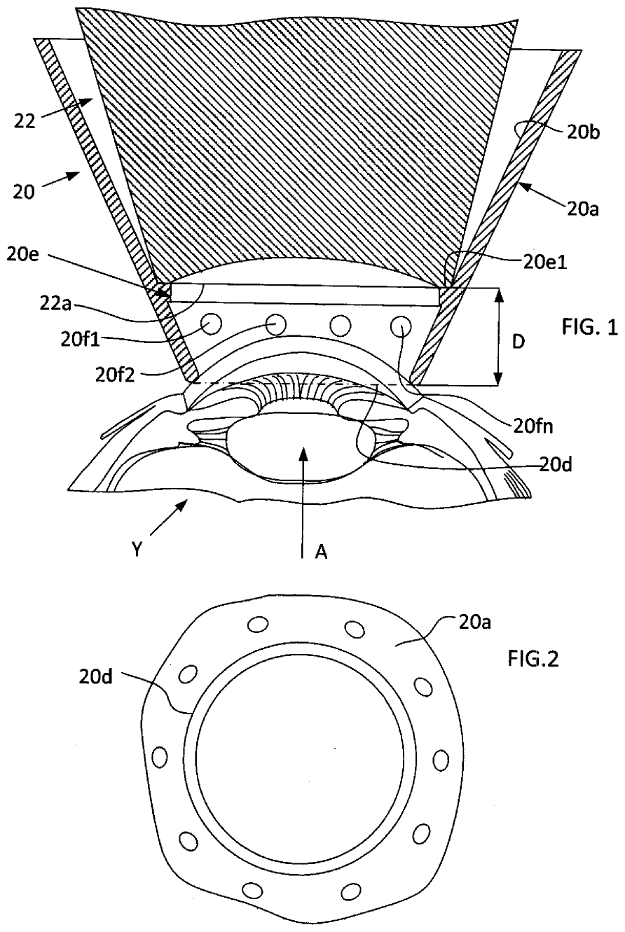

FIG. 1 is s vertical sectional view of a disposable sterile protective cover 20 of the invention (hereinafter referred to merely as a “protective cover”). FIG. 2 is a view of the device of FIG. 1 in the direction of arrow A in FIG. 1.

The protective cover 20 is intended for use in ophthalmology, is made from a resilient material, and has a cup-shaped configuration with a side wall 20a having an inner surface 20b, an open upper endface 20c, and an open lower endface 20d.

For preventing physical contact of the optical surface 22a of an ophthalmic optical device, e.g., a gonio prism 22, with a patient's eye Y at the open lower endface 20d of the protective cover, the cover is provided with an annular shoulder 20e formed on the inner surface 20b of the side wall 20a at a certain distance D distance from the lower endface 20d. The distance D is a distance from the upper surface 20e1 of the shoulder 20e, which supports the optical surface 22a of the gonio prism 22 to the lower endface 20d of the protective cover. To prevent the possibility of transmission of infection, if present, from the eye to the instrument or from the instrument to the eye, through the air gap between the optical surface and the cornea of the eye, the distance D should be in the range of from 0.5 mm to 1.5 MM.

Although in a contactless relation between the instrument and eye the air gap itself is a barrier for transmission of a non-airborne infection, in case of such a transmission through capillaries in the portion of the inner wall 20b under the shoulder 20e, the shoulder itself forms another irresistible barrier on the way of the infection.

In order to prevent fogging on the optical surface 22a of the gonio prism 22, the protective cover of the modification shown in FIGS. 1 and 2 is provided with small holes 20f1, 20f2 . . . 20fn formed in the inner wall 20b of the protective cover 20 under the shoulder 20e, i.e., between the shoulder 20e and the lower endface 20d.

Although, as can be understood from the view show in FIG. 2, the gonio prism and, hence the protective cover 20, has a substantially round cross section, it is understood that the round cross section is shown only as an example, and that the gonio prism and the protective cover may have any other cross section, e.g., a square cross section.

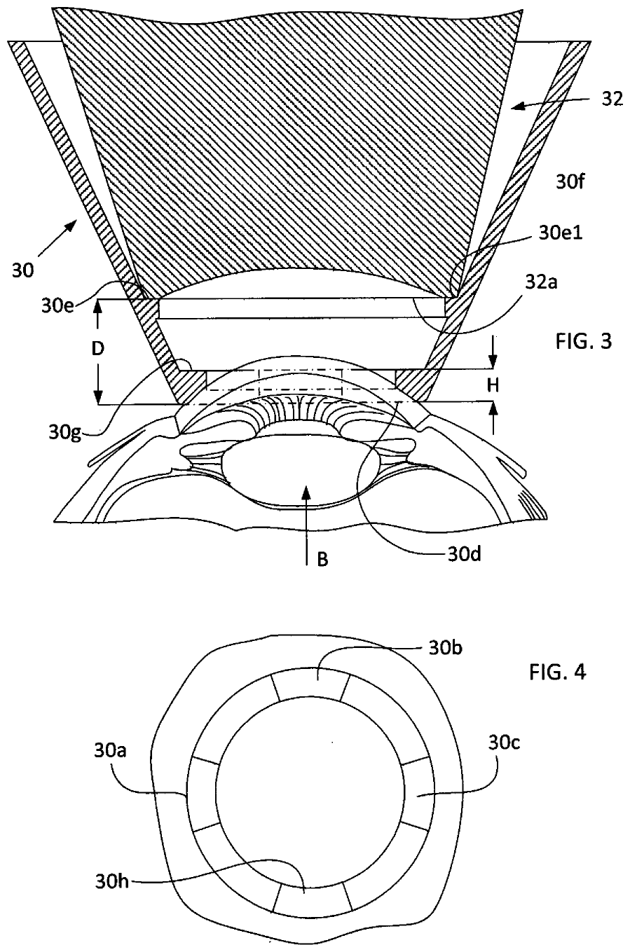

FIG. 3 is a vertical sectional view of a protective cover 30 of the invention in accordance with another modification. FIG. 4 is a view of the protective cover 30 of FIG. 3 in the direction of arrow B in FIG. 3.

The protective cover 30 of the modification shown in FIGS. 3 and 4 is in principle the same as one shown in FIGS. 1 and 2, except that the features for preventing fogging of the optical surface 32a of a gonio prism 32 are projections 30a, 30b, 30c, and 30d formed on the open lower endface 30d of the protective cover 30. The same projections also serve for maintaining and increasing an air gap between the optical surface 32a and the surface of the eye Y1. The projections 30a, 30b, 30c, and 3c are arranged at a circumferentially equal distances. Although four such projections are shown in FIGS. 3 and 4, their number may be different. The projections have a height H from the lower endface 30d to the flat surface 30g in the range of 0.3 mm to 0.8 mm.

Other elements of the protective cover 30, i.e., the side wall 30f, and an annular shoulder 30e are the same as those described and shown above.

The protective covers 20 and 30 can be made from the resilient materials such as polycarbonate, and various commercial plastics based on polymethylmethacrylates, polyurethane, very thin Latex, polystyrene, or the like. and may have a thickness in the range of 0.5 mm to 2.5 mm.

Although the invention was described and illustrated with reference to specific examples, it is understood that that these examples should not be construed as limiting the scope of the invention and that any changes and modifications are possible within the scope of the attached patent claims. For example, as mentioned above, the protective cap may have a cross section that corresponds to the cross section of the optical instrument to be protected from the physical contact with the eye, as well as with any other exposed part of the patient's body. There may be more than three projections on the lower endface of the cover, The surface of the protective cover may be subjected to hydrophobic or any other preliminary treatment. It is understood that the protective cover is supplied in a sterilized form in a sealed package.

1. A disposable sterile protective cover for an optical device having an optical surface and used in ophthalmology, the disposable sterile protective cover being made from a resilient material and having a cup-shaped configuration with a side wall having an inner surface, an open upper endface, and an open lower endface, the disposable sterile protective cover comprising means for preventing physical contact of the optical surface with a patient's eye at the open lower endface, and means for preventing fogging of the optical surface at the open lower endface.

2. The disposable sterile protective cover of claim 1, means for preventing physical contact of the optical surface with the patient's eye is an annular shoulder formed on the inner surface of the side wall, the shoulder having an upper surface at a certain distance from the lower endface of the disposable sterile protective cover.

3. The disposable sterile protective cover of claim 2, wherein the certain distance is selected in the range of from 0.5 mm to 1.5 mm.

4. The disposable sterile protective cover of claim 3, wherein means for preventing fogging of the optical surface at the open lower endface is selected from the group consisting of projections formed on the open lower endface and holes formed in the side wall, which are located below the annular shoulder but above the lower endface.

5. The disposable sterile protective cover of claim 3, wherein the projections are arranged at a circumferentially equal distances.

6. The disposable sterile protective cover of claim 4, wherein the projections are arranged at a circumferentially equal distances.

7. The disposable sterile protective cover of claim 3, wherein the projections have a height from the lower endface in the range of 0.3 mm to 0.8 mm.

8. The disposable sterile protective cover of claim 4, wherein the projections have a height from the lower endface in the range of 0.3 mm to 0.8 mm.

9. The disposable sterile protective cover of claim 6, wherein the projections have a height from the lower endface in the range of 0.3 mm to 0.8 mm.

10. The disposable sterile protective cover of claim 3, wherein the holes formed in the side wall have a diameter in the range of 0.3 mm to 0.8 mm.

11. The disposable sterile protective cover of claim 1, wherein the resilient material of the disposable sterile protective has a thickness in the range of 0.5 mm to 2.5 mm.

12. The disposable sterile protective cover of claim 3, wherein the disposable sterile protective cover is made from a material selected from the group consisting of polycarbonate and polymethylmethacrylates.

13. The disposable sterile protective cover of claim 5, wherein the disposable sterile protective cover is made from a material selected from the group consisting of polycarbonate and polymethylmethacrylates.

14. The disposable sterile protective cover of claim 1, wherein the disposable sterile protective cover is made from a material selected from the group consisting of polycarbonate and polymethylmethacrylates.

15. The disposable sterile protective cover of claim 1, wherein the disposable sterile protective cover is made from a material selected from the group consisting of polycarbonate and polymethylmethacrylates.

16. The disposable sterile protective cover of claim 1, wherein the resilient material of the disposable sterile protective has a thickness in the range of 0.5 mm to 2.5 mm.

17. The disposable sterile protective cover of claim 3, wherein the resilient material of the disposable sterile protective has a thickness in the range of 0.5 mm to 2.5 mm.

18. The disposable sterile protective cover of claim 7, wherein the resilient material of the disposable sterile protective has a thickness in the range of 0.5 mm to 2.5 mm.
